(19) United States
(12) Patent Application Publication
(10) Pub. No.: US 2008/0076728 A1
(43) Pub. Date: Mar. 27, 2008

(54) Novel method of protecting islet cells from apoptosis during the donor harvesting process

(75) Inventors: John E. Thompson, Waterloo (CA); Charles A. Dinarello, Denver, CO (US)

Correspondence Address: WOMBLE CARLYLE SANDRIDGE & RICE, PLLC, ATTN: PATENT DOCKETING 32ND FLOOR, P.O. BOX 7037, ATLANTA, GA, 30357-0037, US

(73) Assignee: Senesco Technologies, Inc., New Brunswick, NJ (US)

(21) Appl. No.: 11/725,470

(22) Filed: Mar. 20, 2007

Related U.S. Application Data
(60) Provisional application No. 60/783,414, filed on Mar. 20, 2006.

(51) Int. Cl. A61K 31/7088; A61P 43/00

(52) U.S. Cl. 514/44

(57) ABSTRACT

The present invention relates to methods for improving the viability and recovery of islets that are separated from a donor organ for subsequent transplantation and more particularly relates to the use of eIF-5A1 siRNAs to enhance the viability of islets.

### RELATED APPLICATIONS

This application claims priority to U.S. Application 60/783,414 filed Mar. 20, 2007, the entire contents of which are incorporated herein.

### BACKGROUND OF THE INVENTION

The islets of Langerhans is a multi-cellular entity containing cells that produce insulin within the pancreas. The average person has about a million islets, and they contain approximately two to three percent of the total number of cells in the pancreas. The pancreas contains the islets of Langerhans, which house beta cells that produce insulin. The beta cells monitor glucose levels in the blood and release finely measured amounts of insulin to counterbalance glucose peaks. Type I and II diabetes develop when more than 90 percent of these beta cells are damaged.

Separation or isolation of the islets from the connective matrix and remaining exocrine tissue is advantageous and beneficial for laboratory experimentation and transplantation purposes. Islet transplantation is a most promising and minimally physiologically invasive procedure for treatment of type I diabetes mellitus. Transplanting islets rather than complete pancreatic tissue has the distinct advantages of ease of transplantation, and the elimination of the pancreatic exocrine function of the donor tissue involving secretion of digestive enzymes. Liberating islets from pancreatic exocrine tissue is the initial and crucial step that influences islet transplantations. The important objective in islet isolations is to provide sufficient numbers of viable functional and potent islets for transplantation.

The “Edmonton Protocol” transplants healthy islets into diabetic patients. Islet transplantation using the Edmonton Protocol is described in Shapiro, Ryan, and Lakey, Clinical Islet Transplantation—State of the Art, Transplantation Proceedings, 33, pp. 3502-3503 (2001); Ryan et al., Clinical Outcomes and Insulin Secretion After Islet Transplantation With the Edmonton Protocol, Diabetes, Vol. 50, April 2001, pp. 710-719; and Ryan et al., Continued Insulin Reserve Provides Long-Term Glycemic Control, Diabetes, Vol. 51, July 2002, pp. 2148-2157. Once in the liver, the cells develop a blood supply and begin producing insulin. The Edmonton Protocol may include 7-10 steps depending on the method employed. The first step involves the delivery of a specific enzyme (liberase) to a donor pancreas, which digests the pancreas tissue, but does not digest the islets. Following the digestion step, there are several successive steps for separating the islets from other cells in the pancreas. The separated islets are transplanted into the main vessel of the liver, known as the portal vein. The liver is able to regenerate itself when damaged, building new blood vessels and supporting tissue. Therefore, when islets are transplanted into the liver, it is believed that new blood vessels form to support the islets. The insulin that the cells produce is absorbed into the blood stream through these surrounding vessels and distributed through the body to control glucose levels in the blood.

Altogether, the steps of the Edmonton Protocol create a vigorous process that compromises the viability of islets, which have a fragile, three-dimensional structure and require large amounts of oxygen for growth and viability. During the process, islets may be damaged or destroyed due to non-optimal conditions of oxygen delivery, affecting the yield of healthy islets that are retrieved from a given donor pancreas. Furthermore, islet transplantation is severely limited by donor availability; frequently, two pancreata are required to obtain insulin independence in just one patient.

Islet transplantation, together with steroid-free, nondiabetogenic immunosuppressive therapy, has been used to treat patients with type 1 diabetes. However, such treatments can lead to increased risk of hyperlipidemia and hypertension, and long-term studies demonstrate that islet viability is impaired.

As a result, there is a need for a method of protecting islet cells from apoptosis during the harvesting process. The present invention provides this need.

### SUMMARY OF THE INVENTION

The present invention provides a method for inhibiting islet cells from undergoing apoptosis during a donor harvesting process comprising administering eIF-5A1 siRNA to the islet cells of an islet cell donor prior to islet isolation, wherein the eIF-5A1 siRNA inhibits expression of eIF-5A1 in the islet cells and thereby inhibits apoptosis in the islet cells. Any siRNA or antisense construct can be used, as long as such construct inhibits expression of eIF-5A1. A preferred siRNA comprises the nucleotide sequence CGGAAUGACUUCCAGCUGAdTdT.

Administration of siRNA may be by any suitable route. Exemplary administration methods include perfusion through the portal vein of the islet cell donor and hydrodynamic perfusion through the portal vein of the islet cell donor.

The present invention also provides a method for inhibiting expression of eIF-5A1 in islet cells comprising administering eIF-5A1 siRNA to the islet cells, wherein the eIF-5A1 siRNA inhibits expression of eIF-5A1 in the islet cells.

Another embodiment of the invention provides a method for inhibiting apoptosis in harvested islet cells comprising administering eIF-5A1 siRNA to the islet cells, wherein the eIF-5A1 siRNA inhibits expression of eIF-5A1 in the islet cells and wherein the inhibition of eIF-5A1 expression inhibits apoptosis.

The present invention also provides a composition for inhibiting apoptosis in islet cells, comprising eIF-5A1 siRNA, wherein the siRNA inhibits expression of eIF-5A1 and thereby inhibits apoptosis in the islet cells. A preferred composition comprises eIF-5A1 siRNA comprising the nucleotide sequence AAAGGAAUGACUUCCAGCUGAdTdT.

### BRIEF DESCRIPTION OF THE DRAWINGS

FIG. 1 provides results of RT-PCR performed for β-actin, mAAT and eIF-5A1 after perfusion through the portal vein with eIF-5A1 siRNA. This figure shows that eIF-5A1 expression is measurable and was thus incorporated into islets.

FIG. 2 shows slow retrograde portal vein perfusion. Bile duct (clear) and portal vein (red) ready for preparatory knot (dark suture). The needle enters below the knot (direction indicated by arrow), crosses under the knot and releases siRNA into vessels that reach pancreas, spleen, intestine and a third of distal colon.

FIG. 3 shows that perfusion of eIF-5A1 siRNA into islets causes a reduction of expression of eIF-5A1 (shown is reduction in mRNA levels of eIF-5A1).

FIG. 4 shows a reduction of apoptosis of islets cells having been treated with eIF-5A1 siRNA as compared to control and saline treated islets (here n=2 per group).

FIG. 5 shows a reduction of apoptosis of islets cells having been treated with eIF-5A1 siRNA as compared to control and saline treated islets (here n=3 per group).

FIG. 6 provides the nucleotide sequence of human eIF-5A1 aligned against eIF-5A2.

FIG. 7 provides the amino acid sequence of human eIF-5A1 aligned against eIF-5A2.

FIG. 8 provides the nucleotide sequence of human eIF-5A1 with exemplary antisense oligonucleotides.

FIG. 9 provides the nucleotide sequence of human eIF-5A1 with exemplary antisense oligonucleotides.

FIGS. 10A and B provide the nucleotide sequence of human eIF-5A1 with exemplary siRNAs.

FIG. 11 provides the nucleotide sequence of human eIF-5A1 with exemplary siRNAs.

### DETAILED DESCRIPTION OF THE INVENTION

It has been previously shown that siRNA incorporation into islets can be achieved by pancreatic perfusion via retrograde portal vein inoculation. See Bradley, et al., Transplantation Proceedings, 37, 233-236, 2005. Briefly, Cy-3 labeled Luciferase (Luc) siRNA GL2 duplex was used either packaged with Lipofectamine 2000 or unpackaged, and injected either through tail vein (in vivo, 50 μg per mouse) or directly into the pancreas by retrograde portal vein inoculation (in situ, 2 μg per mouse). Pancreata were procured and stored at 4° C. for 24 hours after in situ delivery, or 4 hours after in vivo delivery, and islets were isolated and cultured an extra 16 hours before examination. To visualize siRNA distribution, pancreata were stained for insulin and examined under a fluorescent microscope. Isolated islets were directly examined under a fluorescent microscope. Unpackaged siRNA reached islets to a similar extent as observed using liposomal-packaged siRNA, agreeing with reports of so-called “naked”-siRNA delivery in vivo. Lewis et al., Nat. Genet. 32:107-108, Epub 2002 July 2029, 2002 and McCaffrey A P, et al., Nature 418:38-39, 2002).

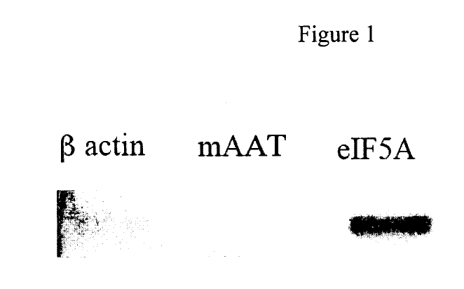

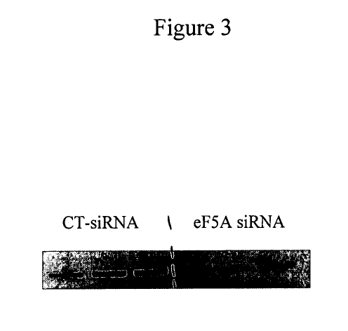

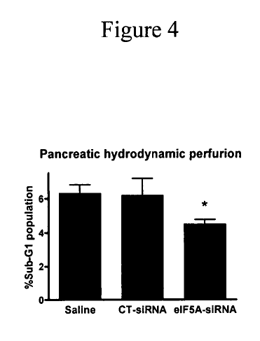

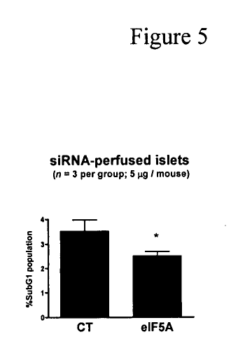

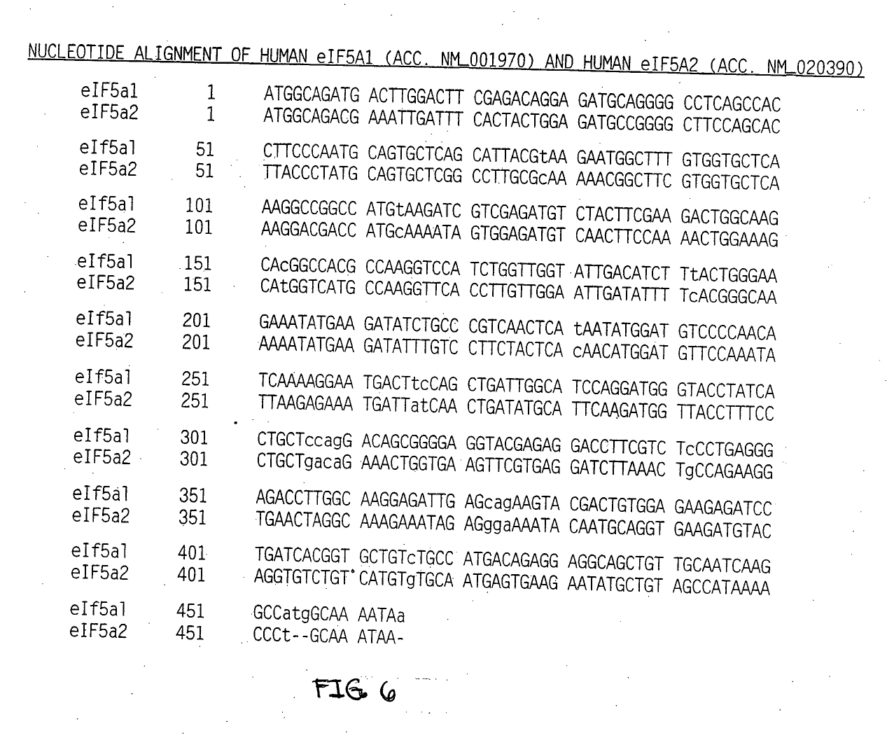

The present invention provides a method for inhibiting expression of eIF-5A1 in islet cells comprising administering eIF-5A1 siRNA to the islet cells, wherein the eIF-5A1 siRNA inhibits expression of eIF-5A1 in the islet cells. FIG. 1 shows that perfusion to the islet cells provides a suitable delivery mechanism to the islet cells and FIG. 3 shows that the eIF-5A1 siRNA treated islet cells do indeed express less eIF-5A1 siRNA. By inhibiting eIF-5A1 expression, apoptosis is also inhibited. FIGS. 4 and 5 shows that treating islets cells with eIF-5A1 siRNA prior to isolation, inhibited these cells from apoptosis (as demonstrated by a reduction of the number of cells in the sub-GI phase). Accordingly, the present invention also provides a method for inhibiting apoptosis in harvested islet cells comprising administering eIF-5A1 siRNA to the islet cells, wherein the eIF-5A1 siRNA inhibits expression of eIF-5A1 in the islet cells and wherein the inhibition of eIF-5A1 expression inhibits apoptosis.

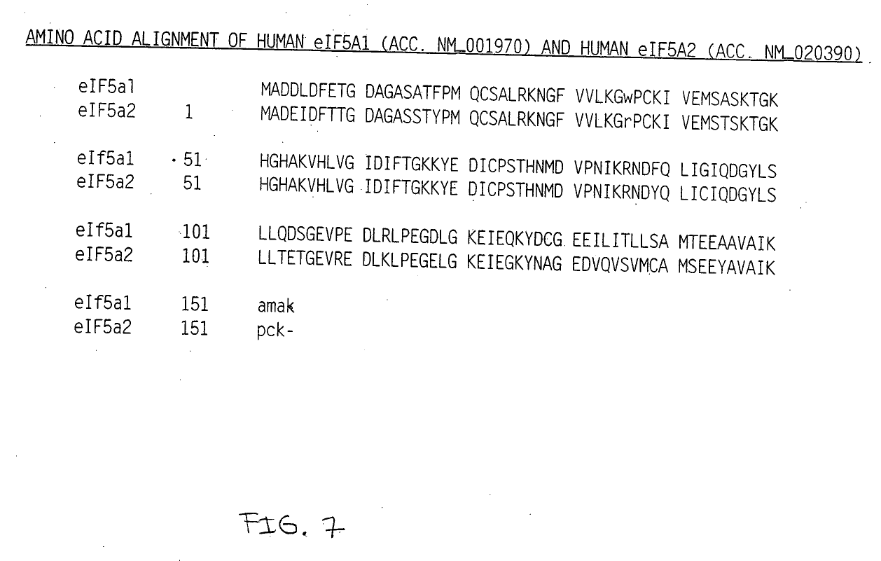

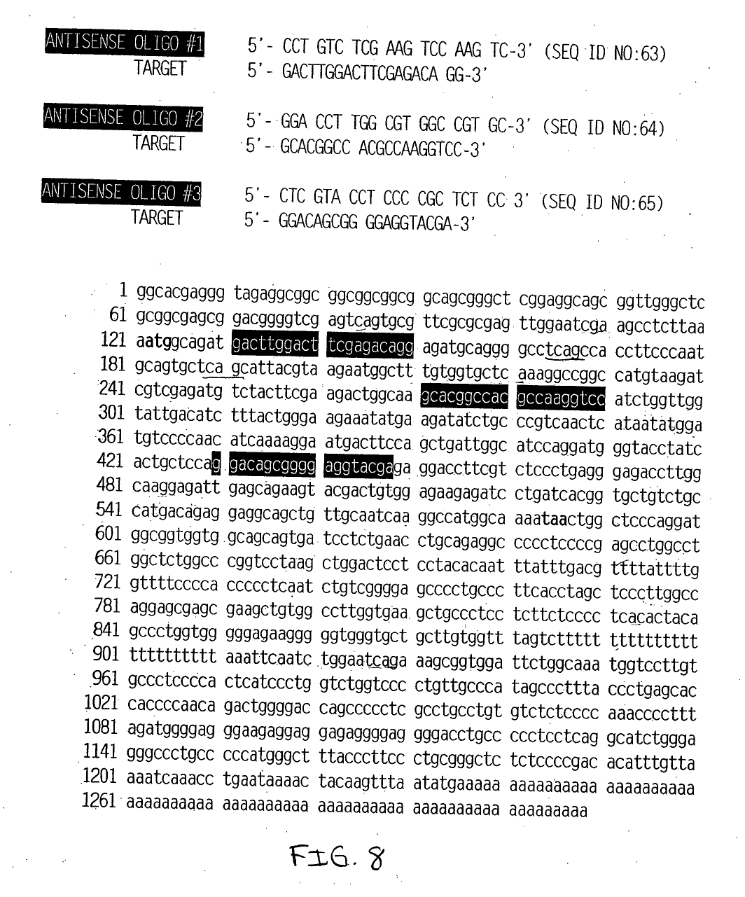

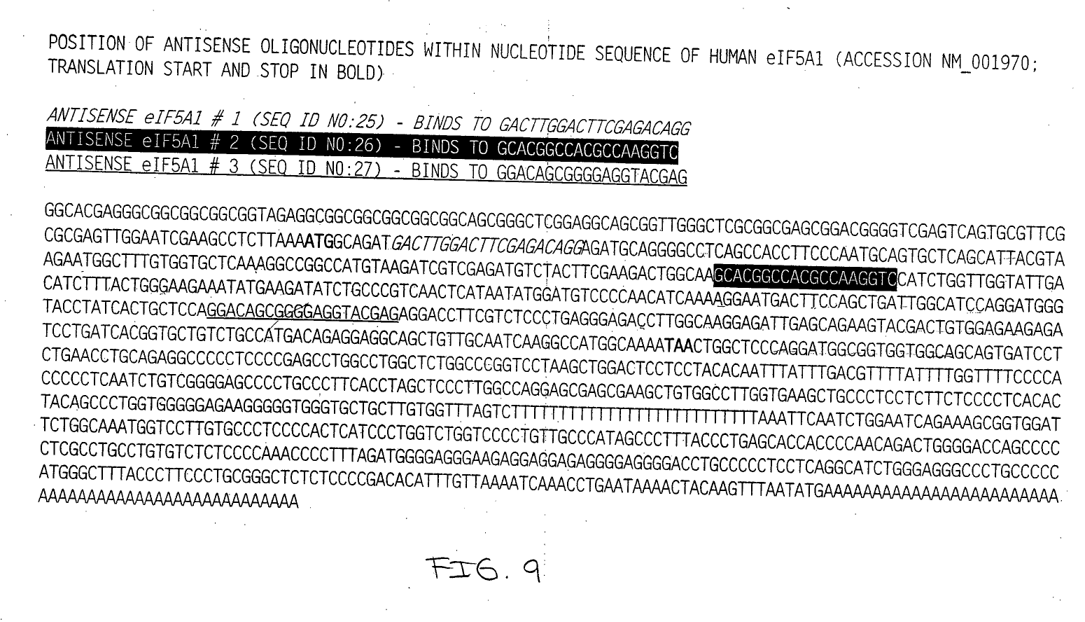

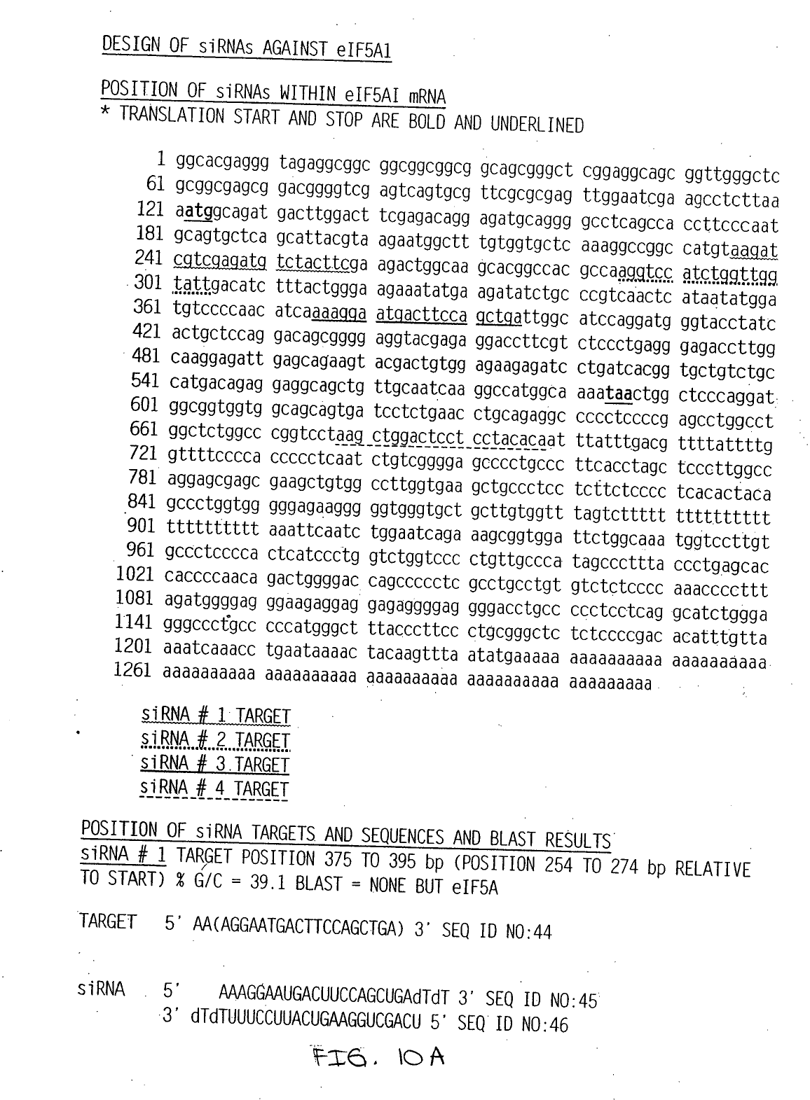

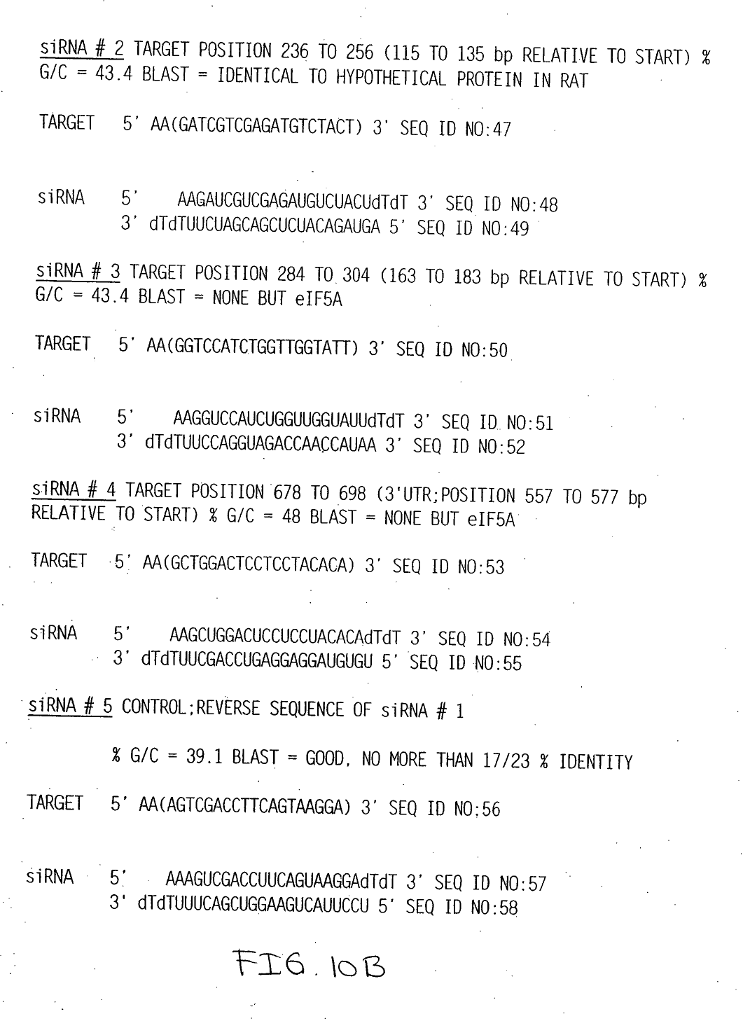

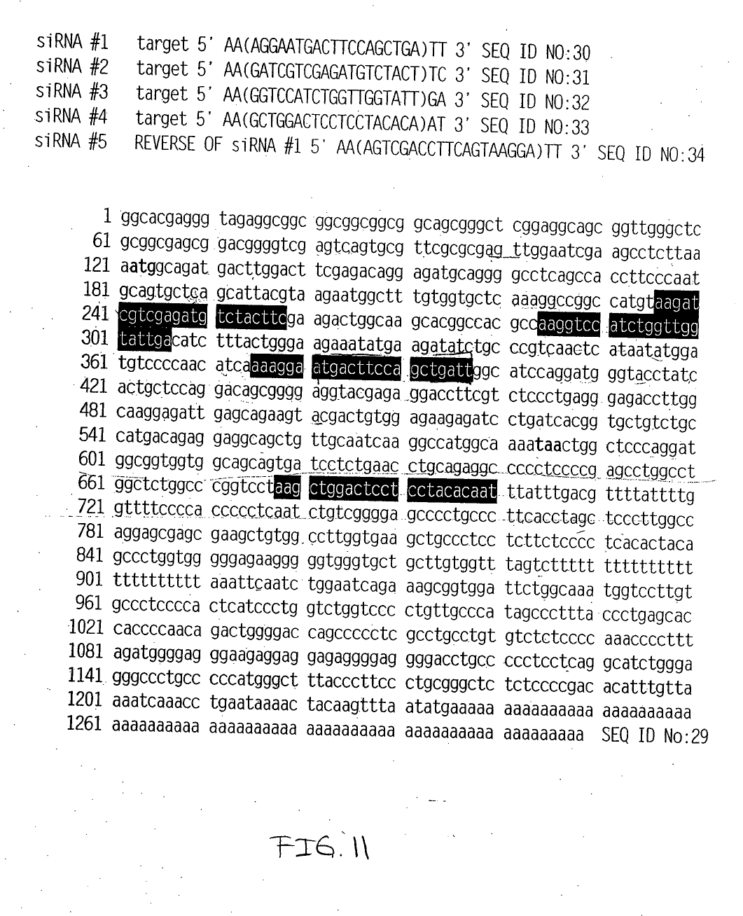

Any eIF-5A1 siRNA that inhibits expression of eIF-5A1 may be used. The term “inhibits” also means reduce. One exemplary eIF-5A1 siRNA comprises the sequence: AAGAUCGUCGAGAUGUCUACUdTdT. Co-pending application Ser. No. 11/293,391, which was filed on Nov. 28, 2005 (which is herein incorporated by reference in its entirety) provides additional exemplary eIF-5A1 siRNAs and other antisense constructs that have been used to inhibit expression of eIF-5A1 in other cell types and were also shown to inhibit apoptosis. One skilled in the art could design other eIF-5A1 siRNAs given the eIF-5A1 sequence and can easily test for the siRNAs ability to inhibit expression without undue experimentation. FIGS. 6-11 provide sequences of eIF-5A1, exemplary eIF-5A1 siRNAs and antisense constructs. In another embodiment of the invention, antisense constructs of eIF-5A1 may be used to inhibit expression of eIF-5A1 and thus inhibit apoptosis of the islet cells.

In preferred embodiments the eIF-5A1 siRNA comprises the nucleotide sequence AAGGUCCAUCUGGUUGGUAUUdTdT.

The present invention also provides a method for inhibiting islet cells from undergoing apoptosis during a donor harvesting process. As discussed above, many islets cells undergo apoptosis when they are harvested. The present inventors have shown that providing eIF-5A1 siRNA to the islet cells prior to harvesting, offers a protective benefit against apoptosis. The eIF-5A1 siRNA is administered to the islet cells of an islet cell donor prior to islet isolation. The donor (and hence islet cells) may be any animal, including human islet cells. Any method of administration may be used. For example, the siRNA may be administered via perfusion through the portal vein of the islet cell donor or via hydrodynamic perfusion through the portal vein of the islet cell donor.

Perfusion through portal vein is similar to canulation of the bile duct, but the needle points the opposite way. The portal vein is exposed by retraction of liver and shifting of visceral organs to the mouse's left. A preparative knot is made around it and includes the bile duct. After puncturing the vessel a blunted needle is advanced toward the pancreas and the knot is tightened around it. In a mouse model, 1 ml saline or siRNA (5 μg) is released slowly, the needle is removed and the knot is closed behind the needle to prevent fluid escape. At this point the mouse is turned around and the bile duct accessed for pancreas digestion. The pancreas may be held longer with siRNA. Alternatively, it can be removed but kept cold with collagenase longer. Regular islet isolation methods are followed and the islets (50) may be incubated in for 16 hours.

The present invention also provides a composition for inhibiting apoptosis in islet cells, comprising eIF-5A1 siRNA, wherein the siRNA inhibits expression of eIF-5A1 and thereby inhibits apoptosis in the islet cells. The composition may comprise other or additional eIF-5A1 siRNAs as discussed above. A preferred siRNA comprises the nucleotide sequence AAGGUCCAUCUGGUUGGUAUUdTdT.

### EXAMPLES

### Mouse Islets Express eIF-5A1

Total RNA was extracted from isolated mouse islets and RT-PCR was performed for (β-actin and for eIF-5A1 (FIG. 1). Resting non-stimulated islets exhibited positive levels of eIF-5A1 mRNA.

eIF-5A1-mRNA Levels Diminished After eIF-5A1-siRNA Delivery: Portal Vein Slow Perfusion.

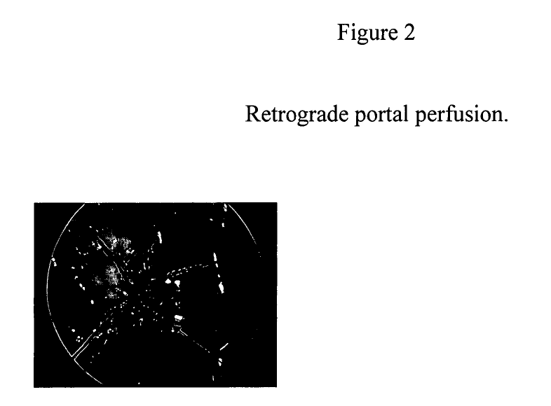

Mice were introduced 1 ml of siRNA (CT (control) sequence or eIF-5A1, 5 μg) or saline, n=2 per group, by slow retrograde portal vein perfusion (FIG. 2). Pancreata were digested by collagenase irrigation of pancreatic duct and islets were isolated as described by Lewis et al., Proc. Natl. Acad. Sci. USA, 102:12153-12158 Epub 12005 Aug. 12110, 2005. Islets (50 per mouse) were incubated for 16 hours. Total RNA was then extracted and RT-PCR was performed for β-actin and for eIF-5A1 (FIG. 3). Ratio of mRNA for eIF-5A1/β-actin was 5.24 (CT-siRNA) and 3.01 eIF-5A1-siRNA). FIG. 3 shows that mRNA levels of eIF-5A1 were reduced in those cells treated with siRNA. This experiment was repeated with n=3 mice and islets were incubated for RNA extraction in triplicates; results were consistent with initial observation.

eIF-5A1-mRNA Levels Diminished and Islet Apoptosis Rate Reduced After eIF-5A1-siRNA Delivery: Portal Vein Hydrodynamic Perfusion.

Mice were introduced 1 ml of siRNA (CT or eIF-5A1, 5 μg) or saline, n=2 per group, by hydrodynamic retrograde portal vein perfusion, which was completed within 5 seconds. Pancreata were digested by collagenase irrigation of pancreatic duct and islets were isolated. Islets were incubated for 16 hours and then divided: one group was stained with propidium iodide for evaluation of apoptosis (50 islets per mouse) and the other group was processed for RT-PCR (25 islets per mouse). Levels of mRNA for eIF-5A1/β-actin were again higher in CT-siRNA group than in eIF-5A1-siRNA group. Apoptosis rate was reduced by 28.1% (FIG. 4). This experiment was repeated with n=3, apoptosis rate again diminished (FIG. 5).

Islets Perfusion with Biotinylated-siRNA.

Biotinylated-siRNA (50 μg) was perfused into islets as described above (slow perfusion, n=1). Pancreas was fixed in formalin for staining.

siRNA.

siRNA molecules were synthesized by Dharmacon, Lafayette, Colo. The sequence of the eIF-5A1 and control siRNA were: 5′ AAAGGAAUGACUUCCAGCUGAdTdT 3′ and 5′ AGUCGACCUUCAGUAAGGCdTdT 3′, respectively.

RT-PCR.

Total RNA was extracted from cells using Qiagen RNeasy kit. eIF-5A1 Primers: Forward 5′-GAC AGT GGG GAG GTA CGA GA-3′; Reverse 5′-GGG GTG AGG AAA ACC AAA AT-3′.

Propidium Iodide (PI) Apoptosis Stain.

Single cell suspension of islets was achieved by gentle trypsinization. Cells were washed with PBS and added saponin-PI mixture containing 0.3% Saponin, EDTA 1 mM, Rnase, 1% Azide, 1% FCS and 50 μg/ml PI in PBS. Cells were thoroughly vortexed and incubated at 4° C. in the dark for 6 hours before analyzed for sub-GI population by FACS.

1. A method for inhibiting islet cells from undergoing apoptosis during a donor harvesting process comprising administering eIF-5A1 siRNA to the islet cells of an islet donor prior to islet isolation wherein the eIF-5A1 siRNA inhibits expression of eIF-5A1 in the islet cells and thereby inhibits apoptosis in the islet cells.

2. The method of claim 1 wherein the eIF-5A 1 siRNA comprises the nucleotide sequence AAAGGAAUGACUUCCAGCUGAdTdT (SEQ ID NO: 2), AAGAUCGUCGAGAUGUCUACUdTdT (SEQ ID NO: 3), AAGGUCCAUCUGGUUGGUAUUdTdT (SEQ ID NO: 4) AAGCUGGACUCCUCCUACACAdTdT (SEQ ID NO: 8), or CGGAAUGACUUCCAGCUGAdTdT (SEQ ID NO: 1).

3. The method of claim 1 wherein the siRNA is administered via perfusion through the portal vein of the islet cell donor.

4. The method of claim 1 wherein the siRNA is administered via hydrodynamic perfusion through the portal vein of the islet cell donor.

5. A method for inhibiting expression of eIF-5A1 in islet cells comprising administering eIF-5A1 siRNA to the islet cells, wherein the eIF-5A1 siRNA inhibits expression of eIF-5A1 in the islet cells.

6. A method for inhibiting apoptosis in harvested islet cells comprising administering eIF-5A1 siRNA to the islet cells, wherein the eIF-5A1 siRNA inhibits expression of eIF-5A1 in the islet cells and wherein the inhibition of eIF-5A1 expression inhibits apoptosis.

7. A composition for inhibiting apoptosis in islet cells, comprising eIF-5A1 siRNA, wherein the siRNA inhibits expression of eIF-5A1 and thereby inhibits apoptosis in the islet cells.

8. The composition of claim 7 wherein the siRNA comprises the nucleotide sequence AAAGGAAUGACUUCCAGCUGAdTdT (SEQ ID NO: 2).

9. The composition of claim 1 wherein the eIF-5A1 siRNA comprises the nucleotide sequence AAAGGAAUGACUUCCAGCUGAdTdT (SEQ ID NO: 2).

10. The method of claim 1 wherein the eIF-5A1 siRNA comprises the nucleotide sequence AAGAUCGUCGAGAUGUCUACUdTdT (SEQ ID NO: 3).

11. The method of claim 1 wherein the eIF-5A1 siRNA comprises the nucleotide sequence AAGGUCCAUCUGGUUGGUAUUdTdT (SEQ ID NO: 4).

12. The composition of claim 7 wherein the eIF-5A1 siRNA comprises the nucleotide sequence AAGCUGGACUCCUCCUACACAdTdT (SEQ ID NO: 8).

13. The composition of claim 7 wherein the eIF-5A1 siRNA comprises the nucleotide sequence AAGGUCCAUCUGGUUGGUAUUdTdT (SEQ ID NO: 4).

14. The composition of claim 7 wherein the eIF-5A1 siRNA comprises the nucleotide sequence CGGAAUGACUUCCAGCUGAdTdT (SEQ ID NO: 1).

15. The method of claim 1 wherein the eIF-5A1 siRNA targets the following nucleotide sequences of eIF-5A1: 5′-AAAGGAATGACTTCCAGCTGA-3′ (SEQ ID NO: 9); 5′-AAGATCGTCGAGATGTCTACT-3′ (SEQ ID NO: 10); 5′-AAGGTCCATCTGGTTGGTATT-3′ (SEQ ID NO: 11); or 5′-AAGCTGGATCCCTCCTACACA-3′ (SEQ ID NO: 12).
